(19) United States
(12) Patent Application Publication
(10) Pub. No.: US 2022/0002306 A1
(43) Pub. Date: Jan. 6, 2022

(54) CRYSTAL FORM OF UPADACITINIB AND PREPARATION METHOD AND USE THEREOF

(71) Applicant: CRYSTAL PHARMACEUTICAL (SUZHOU) CO., LTD., Suzhou , Jiangsu (CN)

(72) Inventors: Jiajia LIU, Suzhou (CN); Jing ZHANG, Suzhou (CN); Min LUO, Suzhou (CN)

(21) Appl. No.: 17/279,471

(22) PCT Filed: Sep. 29, 2019
(86) PCT No.: PCT/CN2019/108835

§ 371 Date: Mar. 24, 2021

(30) Foreign Application Priority Data
Sep. 29, 2018 (CN) 201811150490.8

(51) Int. Cl. C07D 487/14

(52) U.S. Cl. CPC C07D 487/14; C07B 2200/13

(57) ABSTRACT

The present disclosure relates to a crystalline form of upadacitinib and processes for preparation thereof. The present disclosure also relates to a pharmaceutical composition containing the crystalline form, use of the crystalline form for preparing a JAK inhibitor drug, and use of the crystalline form for preparing drugs treating rheumatoid arthritis. The crystalline form of upadacitinib provided by the present disclosure has one or more improved properties compared with prior art and has significant values for future drug optimization and development.

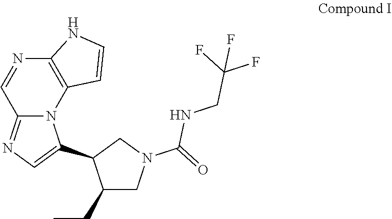 [SMILES: CC[C@@H]1CN(C(=O)NCC(F)(F)F)C[C@@H]1c1cnc2cnc3[nH]ccc3n12]

### TECHNICAL FIELD

The present disclosure relates to the field of pharmaceutical chemistry, particularly relates to a crystalline form of upadacitinib and processes for preparation and use thereof.

### BACKGROUND

Rheumatoid arthritis is an autoimmune disease that can cause chronic inflammation in joints and other parts of the body and leads to permanent joint damage and deformities. If not treated, rheumatoid arthritis can lead to substantial disability and pain due to the damage of joint function, which ultimately leads to shorter life expectancy. JAK1 is a target for immune-inflammatory diseases, and its inhibitors are beneficial for the treatment of rheumatoid arthritis.

Upadacitinib is a second-generation oral JAK1 inhibitor developed by AbbVie, which has a high selectivity for JAK1. The chemical name of upadacitinib is (3S,4R)-3-ethyl-4-(3H-imidazo[1,2-a]pyrrolo[2,3-e]pyrazine-8-yl)-N-(2,2,2-trifluoroethyl) pyrrolidine-1-carboxamide (hereinafter referred to as “Compound I”), and the structure is shown as follows:

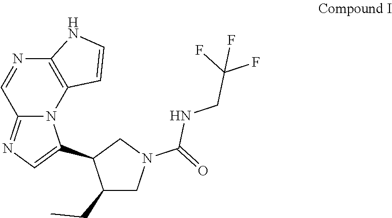 [SMILES: CC[C@@H]1CN(C(=O)NCC(F)(F)F)C[C@@H]1c1cnc2cnc3[nH]ccc3n12]

A crystal is a solid material whose constituents are arranged in a microscopic structure with regular three-dimensional pattern. Polymorphism is the ability of a compound to exist in two or more than two crystalline forms. Different crystalline forms have different physicochemical properties and different in vivo dissolution and absorption, which will further affect drug's clinical efficacy and safety to some extent. In particular, for poorly soluble drugs, the above effects of the crystalline form will be greater. Therefore, drug polymorphism is an important part of drug research and an important part of drug quality control.

WO2017066775A1 disclosed upadacitinib freebase Form A, Form B, Form C, Form D, amorphous and salts thereof. This patent application disclosed that Form A and Form B have poor crystallinity and stability, and can be easily dehydrated to form amorphous. Form D can only be obtained at low water activity. In addition, Form D crystallizes slowly and is hard to reproduce. Form D will convert to Form C at high water activity. Form C is difficult to crystallize from a solution.

As the molecules in the amorphous solids are randomly arranged, they are in a thermodynamically unstable state. Amorphous solids are in a high-energy state, and usually have poor stability. During the manufacturing process and storage, amorphous drugs are prone to crystal transformation, which leads to the change of drug bioavailability, dissolution rate, etc., resulting in changes in the drug's clinical efficacy. In addition, the preparation of amorphous is usually a rapid precipitation process to produce kinetically stable solid, which easily leads to excessive residual solvent. The particle property of amorphous is difficult to control in the preparation process, making it a great challenge in the practical application of drugs.

In order to overcome the disadvantages of the prior art, the inventors of the present disclosure surprisingly discovered crystalline form CSI of compound I. Crystalline form CSI has advantages in at least one aspect of stability, melting point, solubility, in vitro and in vivo dissolution, hygroscopicity, bioavailability, adhesiveness, compressibility, flowability, processability, purification ability, formulation production, etc. In particular, crystalline form CSI has advantages in solubility, intrinsic dissolution rate, dissolution, stability, particle size distribution and compressibility, which provides a new and better choice for the development of upadacitinib and is of great significance.

### SUMMARY OF THE INVENTION

The main objective of the present disclosure is to provide a novel crystalline form of upadacitinib, processes for preparation and use thereof.

According to the objective of the present disclosure, crystalline form CSI of compound I is provided (hereinafter referred to as Form CSI).

According to one aspect of the present disclosure, the X-ray powder diffraction pattern of Form CSI shows characteristic peaks at 2theta values of 10.9°±0.2°, 13.0°±0.2° and 22.9°±0.2° using CuKα radiation.

Furthermore, the X-ray powder diffraction pattern of Form CSI shows one or two or three characteristic peaks at 2theta values of 27.2°±0.2°, 22.3°±0.2° and 16.3°±0.2°. Preferably, the X-ray powder diffraction pattern of Form CSI shows three characteristic peaks at 2theta values of 27.2°±0.2°, 22.3°±0.2° and 16.3°±0.2°.

Further, the X-ray powder diffraction pattern of Form CSI shows one or two or three characteristic peaks at 2theta values of 21.0°±0.2°, 21.5°±0.2° and 25.3°±0.2°. Preferably, the X-ray powder diffraction pattern of Form CSI shows three characteristic peaks at 2theta values of 21.0°±0.2°, 21.5°±0.2° and 25.3°±0.2°.

According to another aspect of the present disclosure, the X-ray powder diffraction pattern of Form CSI shows three or four or five or six or seven or eight or nine or ten or eleven characteristic peaks at 2theta values of 10.9°±0.2°, 13.0°±0.2°, 22.9°±0.2°, 27.2°±0.2°, 22.3°±0.2°, 16.3°±0.2°, 21.0°±0.2°, 21.5°±0.2°, 25.3°±0.2°, 17.7°±0.2° and 19.4°±0.2°.

Without any limitation being implied, Form CSI is an acetic acid solvate which contains 15-24% mass percent of acetic acid, preferably, 17-23% mass percent of acetic acid.

Without any limitation being implied, the X-ray powder diffraction pattern of Form CSI is substantially as depicted in FIG. 1.

Without any limitation being implied, the differential scanning calorimetry curve of Form CSI is substantially as depicted in FIG. 4. An endothermic peak is at around 80-90° C. (onset temperature), which is a desolvation endothermic peak.

Without any limitation being implied, the thermo gravimetric analysis curve of Form CSI is substantially as depicted in FIG. 3, which shows 15%-24% weight loss when heated to 135±5° C., preferably, 17-23% weight loss when heated to 135±5° C.

According to the objective of the present disclosure, a process for preparing Form CSI is also provided, wherein the process comprises:

Mixing upadacitinib freebase, acetic acid and organic solvents, and stirring to obtain a solid.

Furthermore, said mixing is preferably dissolving upadacitinib freebase in acetic acid, and then mixing with organic solvents; or adding upadacitinib freebase in a mixture of acetic acid and organic solvents.

Furthermore, said organic solvents are preferably ethers or alkanes.

Furthermore, said ether is preferably methyl tert-butyl ether, said alkane is preferably n-hexane, n-heptane and a mixture thereof.

Furthermore, said acetic acid and upadacitinib freebase in a solvent system has a stoichiometric ratio of 3:1-120:1.

Furthermore, said acetic acid and upadacitinib freebase in a solvent system has a stoichiometric ratio of 3:1-10:1.

Form CSI of the present disclosure has the following advantages:

(1) Compared with prior art, Form CSI of the present disclosure has a higher solubility. Compared with prior art, Form CSI has a higher solubility in pH7.4 PBS (Sodium phosphate buffer), pH6.5 FaSSIF (Fasted state simulated intestinal fluids) and pH5.0 FeSSIF (Fed state simulated intestinal fluids). In particular, the solubility of Form CSI in FaSSIF is 18 times that of Form C in prior art WO2017066775A1.

Higher solubility is beneficial to improve drug product's in vivo absorption and bioavailability, thus improving drug efficacy. In addition, drug dose reduction without affecting efficacy is possible due to higher solubility, thereby reducing the side effects of drugs and improving drug safety.

(2) Form CSI of the present disclosure has good in vitro dissolution and dissolution rate. In pH6.8 PBS, intrinsic dissolution rate of Form CSI drug substance is more than 8 times that of Form C in prior art WO2017066775A1. In pH6.8 PBS, dissolution of Form CSI drug product is higher than that of Form C in prior art WO2017066775A1.

Crystalline form difference can affect drug product's in vivo dissolution rates, which directly affects drug's in vivo absorption, distribution, excretion and metabolism, and finally leads to difference in clinical efficacy due to different bioavailability. Dissolution and dissolution rates are important prerequisites for drug absorption. Good in vitro dissolution leads to higher in vivo absorption, better in vivo exposure, thereby improving drug's bioavailability and efficacy. High dissolution rate is beneficial for the drug to achieve peak concentration in plasma quickly after administration, thus ensuring rapid drug action.

(3) Form CSI drug substance of the present disclosure has good stability. Form CSI drug substance remains unchanged for at least six months after being stored under 25° C./60% RH (Relative Humidity). The chemical purity of Form CSI drug substance remains substantially unchanged during storage. These results show that Form CSI has good stability under a long-term condition, which is suitable for drug product storage.

Meanwhile, the crystalline state of Form CSI drug substance remains unchanged for at least 6 months when stored under a condition of 4° C., and remains unchanged for at least two weeks when stored under 40° C./75% RH and 60° C./75% RH. The chemical purity remains substantially unchanged during storage. These results show that Form CSI has good stability under accelerated and more stress conditions. Good stability under accelerated and stress conditions is of great importance to the drug development. Drug substance will go through high temperature and high humidity conditions caused by weather, season and regional climate differences during storage, transportation, and manufacturing processes. Form CSI drug substance has good stability under these stress conditions, which is beneficial to avoid the influence on drug quality when not stored in condition recommended in label. Meanwhile, Form CSI has good mechanical stability. Form CSI drug substance remains unchanged after grinding. Therefore, Form CSI has good physical stability. Grinding and pulverization are often required in formulation process. Good grinding stability of Form CSI can reduce the risk of crystallinity change and crystal transformation in drug substance during formulation process. Form CSI drug substance has good physical stability under different pressure, which is beneficial to keep crystalline form unchanged during tableting process.

Crystal transformation in drug product may lead to changes in the absorption of drugs, affect bioavailability, and even cause toxicity and side effects. Good chemical stability ensures that no impurities are generated during storage. Form CSI has good physicochemical stability, ensuring consistent and controllable quality of the drug substance and drug product, minimizing change in quality, bioavailability, even toxicity and side effects due to crystal transformation or impurity generation.

Furthermore, Form CSI of the present disclosure also have the following advantages:

(1) Compared with prior art, Form CSI of the present disclosure has uniform particle size distribution, which helps to ensure uniformity of content and reduce variability of in vitro dissolution. At the same time, the preparation processes can be simplified, cost can be reduced, and risks of decrease in crystallinity and crystal transformation caused by grinding can be reduced.

(2) Compared with prior art, Form CSI of the present disclosure has better compressibility. Failure in hardness/friability test and tablet crack issue can be avoided due to better compressibility of Form CSI, making process of drug product more reliable, improving product appearance and product quality. Better compressibility increases the compression rate, thus further increases the efficiency of process and reduces the cost of compressibility improving excipients.

According to the objective of the present disclosure, a pharmaceutical composition is also provided. Said pharmaceutical composition comprises a therapeutically effective amount of Form CSI and pharmaceutically acceptable carriers, diluents or excipients.

Furthermore, Form CSI of the present disclosure can be used for preparing JAK inhibitor drugs.

Furthermore, Form CSI of the present disclosure can be used for preparing drugs treating rheumatoid arthritis.

In the present disclosure, said “stirring” is accomplished by using a conventional method in the field such as magnetic stirring or mechanical stirring and the stirring speed is 50 to 1800 r/min, preferably the magnetic stirring speed is 300 to 900 r/min and mechanical stirring speed is 100 to 300 r/min.

Said “drying” is accomplished at room temperature or a higher temperature. The drying temperature is from room temperature to about 60° C., or to 50° C., or to 40° C. The drying time can be 2 to 48 hours, or overnight. Drying is accomplished in a fume hood, forced air convection oven or vacuum oven.

In the present disclosure, “crystal” or “crystalline form” refers to the crystal or the crystalline form being identified by the X-ray diffraction pattern shown herein. Those skilled in the art are able to understand that physicochemical properties discussed herein can be characterized. The experimental errors depend on the instrument conditions, the sample preparation and the purity of samples. In particular, those skilled in the art generally know that the X-ray diffraction pattern typically varies with the experimental conditions. It is necessary to point out that, the relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and identical diffraction peak intensities are not required. In addition, the experimental error of the diffraction peak position is usually 5% or less, and the error of these positions should also be taken into account. An error of ±0.2° is usually allowed. In addition, due to experimental factors such as sample thickness, the overall offset of the diffraction peak is caused, and a certain offset is usually allowed. Thus, it will be understood by those skilled in the art that a crystalline form of the present disclosure is not necessarily to have exactly the same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms. In some embodiments, Form CSI of the present disclosure is pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and furthermore specifically less than 1% (w/w).

In the present disclosure, the term “about” when referring to a measurable value such as weight, time, temperature, and the like, is meant to encompass variations of ±10%, ±5%, ±1%, ±0.5%, or even ±0.1% of the specified amount.

### BRIEF DESCRIPTION OF THE DRAWINGS

FIG. 1 shows an XRPD pattern of Form CSI in Example 1.

FIG. 2 shows a ¹H NMR spectrum of Form CSI in Example 1.

FIG. 3 shows a TGA curve of Form CSI in Example 2.

FIG. 4 shows a DSC curve of Form CSI in Example 4.

FIG. 5 shows an XRPD pattern of Form CSI in Example 6.

FIG. 6 shows an XRPD pattern of Form CSI in Example 7.

FIG. 7 shows a TGA curve of Form CSI in Example 7.

FIG. 8 shows intrinsic dissolution profiles of Form CSI and Form C in prior art WO2017066775A1.

FIG. 9 shows an XRPD pattern overlay of Form CSI before and after storage (from top to bottom:

initial crystalline form, stored under 4° C. (in a closed dish) for 6 months).

FIG. 10 shows an XRPD pattern overlay of Form CSI before and after storage (from top to bottom: initial crystalline form, stored under 25° C./60% RH (in a closed dish) for 6 months).

FIG. 11 shows an XRPD pattern overlay of Form CSI before and after storage (from top to bottom: initial crystalline form, storage under 40° C./75% RH (in a closed dish) for 2 weeks).

FIG. 12 shows an XRPD pattern overlay of Form CSI before and after storage (from top to bottom: initial crystalline form, storage under 60° C./75% RH (in a closed dish) for 2 weeks).

FIG. 13 shows an XRPD pattern overlay of Form CSI before and after tableting (from top to bottom: sample compressed with 14 kN, 7 kN, 3 kN and 0 kN pressure).

FIG. 14 shows an XRPD pattern overlay of Form CSI before and after grinding (from top to bottom: Form CSI before grinding, Form CSI after grinding).

FIG. 15 shows a PSD plot of Form CSI.

FIG. 16 shows a PSD plot of Form C in WO2017066775A1.

FIG. 17 shows an XRPD pattern overlay of Form CSI before and after formulation (from top to bottom: drug product, mixture of excipients, Form CSI).

FIG. 18 shows an XRPD pattern overlay of Form CSI before and after formulation (from top to bottom: drug product, mixture of excipient, Form CSI).

FIG. 19 shows dissolution profiles of Form CSI and Form C in WO2017066775A1.

### DETAILED DESCRIPTION OF PREFERRED EMBODIMENTS

The present disclosure is further illustrated by the following examples which describe the preparation and use of the crystalline form of the present disclosure in detail. It is obvious to those skilled in the art that many changes in the materials and methods can be accomplished without departing from the scope of the present disclosure.

The abbreviations used in the present disclosure are explained as follows:
XRPD: X-Ray Powder Diffraction
DSC: Differential Scanning calorimetry
TGA: Thermo Gravimetric Analysis
¹H NMR: Proton Nuclear Magnetic Resonance
HPLC: High Performance Liquid Chromatography
PSD: Particle Size Distribution

Instruments and methods used for data collection:

X-ray powder diffraction patterns in the present disclosure were acquired by a Bruker D2 PHASER X or Bruker D8 Discover X-ray powder diffractometer. The parameters of the X-ray powder diffraction method of the present disclosure are as follows:
X-Ray Reflection: Cu, Kα
Kα1 (Å): 1.54060; Kα2 (Å): 1.54439
Kα2/Kα1 intensity ratio: 0.50

Differential scanning calorimetry (DSC) data in the present disclosure were acquired by a TA Q2000. The parameters of the DSC method of the present disclosure are as follows:
Heating rate: 10° C./min
Purge gas: nitrogen

Thermogravimetric analysis (TGA) data in the present disclosure were acquired by a TA Q500. The parameters of the TGA method of the present disclosure are as follows:

Heating rate: 10° C./min

Purge gas: nitrogen

Proton nuclear magnetic resonance (′H NMR) spectrum data were collected from a Bruker Avance II DMX 400M HZ NMR spectrometer. 1-5 mg of sample was weighed and dissolved in 0.5 mL of deuterated dimethyl sulfoxide to obtain a solution with a concentration of 2-10 mg/mL.

The particle size distribution data in the present disclosure were acquired by a Mastersizer 3000 laser particle size analyzer of Malvern. The test is carried out in wet mode by Hydro MV dispersing device, and the dispersion medium is Isopar G. The method parameters of the laser particle size analyzer are as follows:

| | |
|---|---|
| Size distribution: Volume | Run Time: 10 s |
| Dispersion medium: Isopar G | Particle coordinates: Standard |
| Run Number: Average of 3 runs | Fluid refractive index: 1.42 |
| Absorption rate: 0.1 | Residuals: Enabled |
| Particle refractive index: 1.52 | Ultrasonication power: 30 W |
| Particle shape: Irregular | Ultrasonication time: 30 s |

Solubility measurement for acetic acid content in upadacitinib freebase acetic acid solvate of the present disclosure:

| HPLC | Agilent 1260 | |
|---|---|---|
| Column | Waters Xbridge C18, 150 × 4.6 mm, 5 μm | |
| Mobile Phase | A: 0.1% trifluoroacetic acid in water | |
| | B: 0.1% trifluoroacetic acid in acetonitrile | |
| Gradient | Time (min) | % B |
| | 0.0 | 5.0 |
| | 5.0 | 5.0 |
| | 10.0 | 70.0 |
| | 11.0 | 5.0 |
| | 18.0 | 5.0 |

| | |
|---|---|
| Running time | 18.0 min |
| Post running time | 0.0 min |
| Flow rate | 1.0 mL/min |
| Injection Volume | 5 μL |
| Detection wavelength | UV at 210 nm |
| Column Temperature | 40° C. |
| Temperature of Sample | RT |
| Tray | |

Solubility measurement for freebase content in upadacitinib freebase acetic acid solvate of the present disclosure. Solubility measurement for upadacitinib freebase acetic acid solvate of the present disclosure in FaSSIF and FeSSIF:

| HPLC | Agilent 1260 | |
|---|---|---|
| Column | Waters Xbridge C18, 150 × 4.6 mm, 5 μm | |
| Mobile Phase | A: 0.1% trifluoroacetic acid in water | |
| | B: 0.1% trifluoroacetic acid in acetonitrile | |
| Gradient | Time (min) | % B |
| | 0.0 | 15.0 |
| | 7.0 | 80.0 |
| | 10.0 | 80.0 |
| | 10.1 | 15.0 |
| | 15.0 | 15.0 |

| | |
|---|---|
| Running time | 15.0 min |
| Post running time | 0.0 min |
| Flow rate | 1.0 mL/min |
| Injection Volume | 5 μL |
| Detection wavelength | UV at 220 nm |
| Column Temperature | 40° C. |
| Temperature of Sample | RT |
| Tray | |

Solubility measurement for solubility, dissolution, and intrinsic dissolution rate in PBS in the present disclosure:

| HPLC | Agilent 1260 | |
|---|---|---|
| Column | Waters Xbridge C18, 150*4.6 mm, 5 μm | |
| Mobile Phase | A: 0.1% trifluoroacetic acid in water | |
| | B: 0.1% trifluoroacetic acid in acetonitrile | |
| Gradient | Time (min) | % B |
| | 0.0 | 20.0 |
| | 10.0 | 20.0 |

| | |
|---|---|
| Running time | 10.0 min |
| Post running time | 0.0 min |
| Flow rate | 1.0 mL/min |
| Injection Volume | 5 μL (solubility in PBS)/ |
| | 50 μL (intrinsic dissolution rate/dissolution) |
| Detection wavelength | UV at 220 nm |
| Column Temperature | 40° C. |
| Temperature of Sample | RT |
| Tray | |
| Diluent | ACN/H₂O (v/v, 1/1) |

Unless otherwise specified, the following examples were conducted at room temperature. Said “room temperature” is not a specific temperature, but a temperature range of 10−30° C.

According to the present disclosure, upadacitinib and/or its salt used as a raw material is solid (crystalline and amorphous), oil, liquid form or solution. Preferably, compound I and/or its salt used as a raw material is a solid.

Upadacitinib and/or a salt thereof used in the following examples were prepared by known methods in prior art, for example, the method disclosed in WO2017066775A1.

Form C in WO2017066775A1 of the present disclosure was prepared by method A of example 7 disclosed in WO2017066775A1.

### Example 1: Preparation of Form CSI

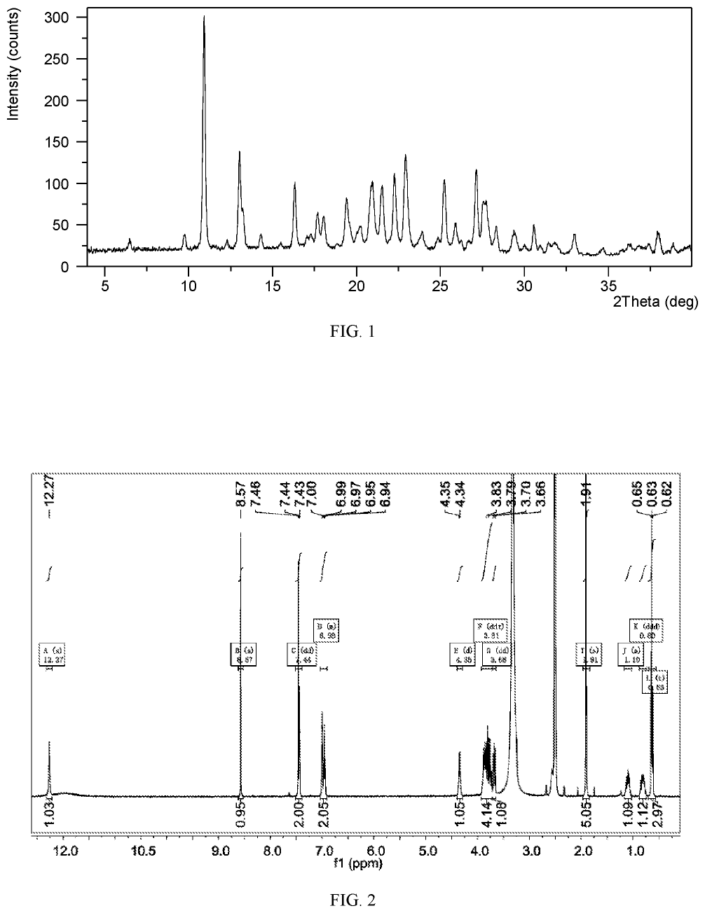

11.2 mg of upadacitinib freebase was weighed into a 4-mL glass vial and dissolved with 0.2 mL of acetic acid. About 5.0 mL of n-hexane was added into a 20-mL glass vial. Then the 4-mL glass vial was placed into this 20-mL glass vial, and the 20-mL glass vial was sealed with screw cap and placed at room temperature for 45 days. The 4-mL glass vial was taken out and 1.0 mL of n-hexane was added into it. Then the system was placed at −20° C. with slurry for 40 days. The solid obtained was isolated and dried under vacuum at room temperature for 5.5 hours. The obtained solid was confirmed to be Form CSI. The XRPD data are listed in Table 1, and the XRPD pattern is as depicted in FIG. 1.

The ¹H NMR spectrum of Form CSI is substantially as depicted in FIG. 2, and the results are in consistent with the structure of compound I (C₁₇H₁₉F₃N₆O). The characteristic peak at 1.91 ppm belongs to acetic acid, which indicates that Form CSI is an acetic acid solvate containing 20.8 wt % of acetic acid. The corresponding data are: ¹H NMR (400 MHz, DMSO-d6) δ 12.27 (s, 1H), 8.57 (s, 1H), 7.44 (dd, J=7.5, 4.4 Hz, 2H), 7.04-6.91 (m, 2H), 4.35 (d, J=6.3 Hz, 1H), 3.81 (ddt, J=16.4, 10.4, 7.8 Hz, 4H), 3.68 (dd, J=10.2, 7.0 Hz, 1H), 1.91 (s, 5H), 1.17-1.02 (m, 1H), 0.80 (ddd, J=16.7, 13.6, 6.9 Hz, 1H), 0.63 (t, J=7.3 Hz, 3H).

TABLE 1

| 2θ | d spacing | Intensity % |
|---|---|---|
| 9.74 | 9.08 | 6.16 |
| 10.93 | 8.10 | 100.00 |
| 13.02 | 6.80 | 42.99 |
| 16.31 | 5.43 | 28.88 |
| 17.66 | 5.02 | 15.85 |
| 18.03 | 4.92 | 14.64 |
| 19.39 | 4.58 | 22.42 |
| 20.16 | 4.40 | 9.75 |
| 20.93 | 4.24 | 30.30 |
| 21.52 | 4.13 | 28.66 |
| 22.25 | 4.00 | 33.72 |
| 22.89 | 3.88 | 41.92 |
| 23.87 | 3.73 | 8.22 |
| 25.23 | 3.53 | 31.81 |
| 27.10 | 3.29 | 36.53 |
| 27.63 | 3.23 | 22.32 |
| 28.31 | 3.15 | 11.61 |
| 29.32 | 3.05 | 8.00 |
| 30.55 | 2.93 | 13.01 |
| 31.70 | 2.82 | 4.05 |
| 32.97 | 2.72 | 8.33 |
| 37.93 | 2.37 | 7.33 |

### Example 2: Preparation of Form CSI

10.1 mg of upadacitinib freebase was dissolved into 0.25 mL of methyl tert-butyl ether/acetic acid (4:1, v/v) at room temperature, followed by the addition of a small amount of sand. The system was cooled to 5° C. at a rate of 0.1° C./min and placed at 5° C. for 12 hours. Then the system was placed at −20° C. for 24 hours and then stirred at −20° C. for 4 days. A solid was obtained after isolation and vacuum drying at room temperature for 6 hours. The obtained solid was confirmed to be Form CSI of the present disclosure by XRPD and the XRPD data are listed in Table 2.

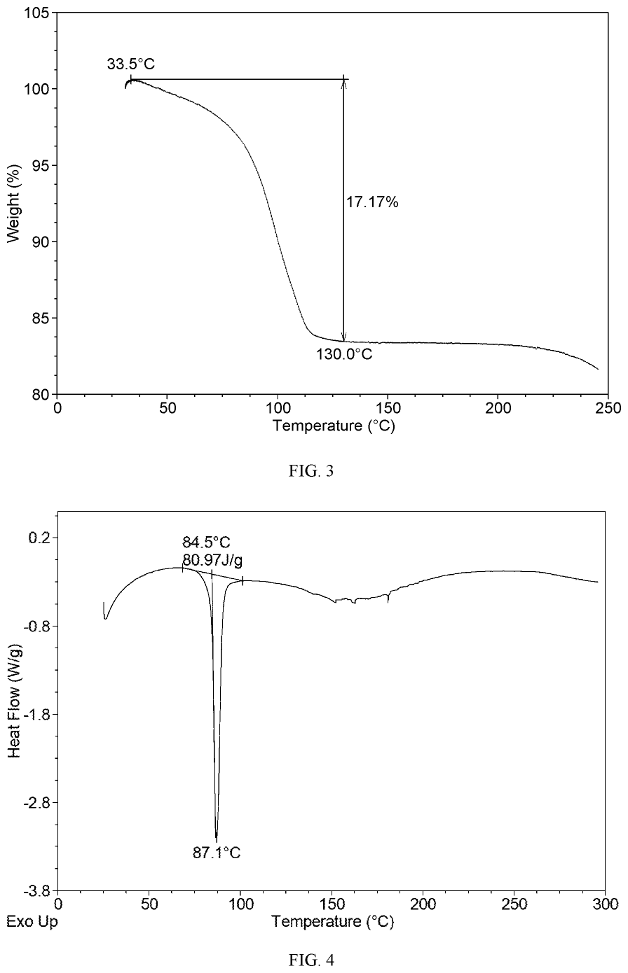

The TGA curve of Form CSI shows about 17.2% weight loss when heated to 130° C., which is substantially as presented in FIG. 3.

TABLE 2

| 2θ | d spacing | Intensity % |
|---|---|---|
| 10.93 | 8.10 | 100.00 |
| 13.03 | 6.79 | 45.21 |
| 16.35 | 5.42 | 28.42 |
| 17.70 | 5.01 | 13.66 |
| 18.05 | 4.92 | 14.97 |
| 19.42 | 4.57 | 17.49 |
| 21.02 | 4.23 | 24.12 |
| 21.53 | 4.13 | 21.75 |
| 22.31 | 3.98 | 21.83 |
| 22.93 | 3.88 | 25.17 |
| 25.25 | 3.53 | 12.80 |
| 26.62 | 3.35 | 24.18 |
| 27.21 | 3.28 | 19.50 |
| 27.48 | 3.25 | 28.17 |
| 27.92 | 3.20 | 14.08 |
| 33.01 | 2.71 | 3.18 |

### Example 3: Preparation of Form CSI

44.3 mg of upadacitinib freebase was dissolved into 0.8 mL of acetic acid and the solution was filtered. 0.27 mL of the filtrate was transferred into a 4-mL glass vial. About 5.0 mL of n-hexane was added into a 20-mL glass vial, and the 4-mL glass vial was placed into this 20-mL glass vial, which was capped and placed at room temperature for nine days. The 4-mL glass vial was taken out and 1.0 mL of n-heptane was added, then the vial was placed at −20° C. with stirring until solid obtained. The solid was isolated and confirmed to be Form CSI by XRPD. The XRPD data are listed in Table 3. Form CSI has about 20.1% weight loss when heated to 130° C.

TABLE 3

| 2θ | d spacing | Intensity % |
|---|---|---|
| 3.45 | 25.64 | 4.91 |
| 10.93 | 8.10 | 100.00 |
| 12.97 | 6.83 | 60.90 |
| 16.25 | 5.46 | 43.46 |
| 17.70 | 5.01 | 56.36 |
| 19.57 | 4.54 | 19.99 |
| 20.28 | 4.38 | 28.11 |
| 21.09 | 4.21 | 31.86 |
| 21.56 | 4.12 | 33.64 |
| 22.30 | 3.99 | 65.81 |
| 22.89 | 3.89 | 33.31 |
| 25.26 | 3.53 | 22.67 |
| 27.15 | 3.28 | 56.80 |
| 27.57 | 3.24 | 24.36 |
| 30.62 | 2.92 | 11.23 |

### Example 4: Preparation of Form CSI

41.9 mg of upadacitinib freebase was weighed into a 5-mL glass vial, then 0.1 mL of acetic acid and 1.0 mL of n-hexane were added to dissolve the upadacitinib freebase with ultrasound and heating. The obtained solution was transferred to a −20° C. environment and stirred for 15 minutes and a small amount of white solid was produced. 1.0 mL of n-hexane was added into this suspension and the suspension was stirred at −20° C. for another 10 days. After isolation and drying, a solid was obtained and confirmed to be Form CSI of the present disclosure by XRPD. The XRPD data are listed in Table 4.

The DSC curve of Form CSI is substantially as depicted in FIG. 4, which shows one endothermic peak at around 85° C. (onset temperature), corresponding to the endothermic process caused by the loss of acetic acid.

TABLE 4

| 2θ | d spacing | Intensity % |
|---|---|---|
| 10.93 | 8.10 | 100.00 |
| 13.04 | 6.79 | 28.12 |
| 14.33 | 6.18 | 4.01 |
| 16.33 | 5.43 | 15.41 |
| 17.71 | 5.01 | 12.55 |
| 18.06 | 4.91 | 10.85 |
| 19.40 | 4.57 | 12.79 |
| 20.17 | 4.40 | 6.76 |
| 21.02 | 4.23 | 23.60 |
| 21.55 | 4.12 | 25.32 |
| 22.27 | 3.99 | 17.44 |
| 22.94 | 3.88 | 16.72 |
| 25.28 | 3.52 | 10.48 |
| 27.15 | 3.28 | 18.26 |
| 27.58 | 3.23 | 11.29 |

### Example 5 Preparation of Form CSI

300.3 mg of upadacitinib freebase was dissolved into 7.5 mL of methyl tert-butyl ether/acetic acid (4:1, v/v) at room temperature, followed by the addition of a small amount of sand. After being stirred at −20° C. for 4 days, the solid was isolated and confirmed to be Form CSI by XRPD. The XRPD data are listed in Table 5.

Form CSI has about 19.3% weight loss when heated to 140° C.

TABLE 5

| 2θ | d spacing | Intensity % |
|---|---|---|
| 10.93 | 8.10 | 100.00 |
| 13.04 | 6.79 | 29.16 |
| 16.33 | 5.43 | 18.41 |
| 17.77 | 4.99 | 20.18 |
| 18.08 | 4.91 | 11.59 |
| 19.42 | 4.57 | 12.90 |
| 20.33 | 4.37 | 13.99 |
| 21.00 | 4.23 | 31.45 |
| 21.54 | 4.12 | 27.02 |
| 22.29 | 3.99 | 18.03 |
| 22.96 | 3.87 | 20.13 |
| 24.03 | 3.70 | 7.70 |
| 25.30 | 3.52 | 13.38 |
| 26.00 | 3.43 | 8.30 |
| 26.69 | 3.34 | 9.75 |
| 27.29 | 3.27 | 28.59 |
| 27.63 | 3.23 | 18.26 |
| 28.41 | 3.14 | 7.41 |
| 29.59 | 3.02 | 6.14 |
| 30.66 | 2.92 | 5.46 |

### Example 6 Preparation of Form CSI

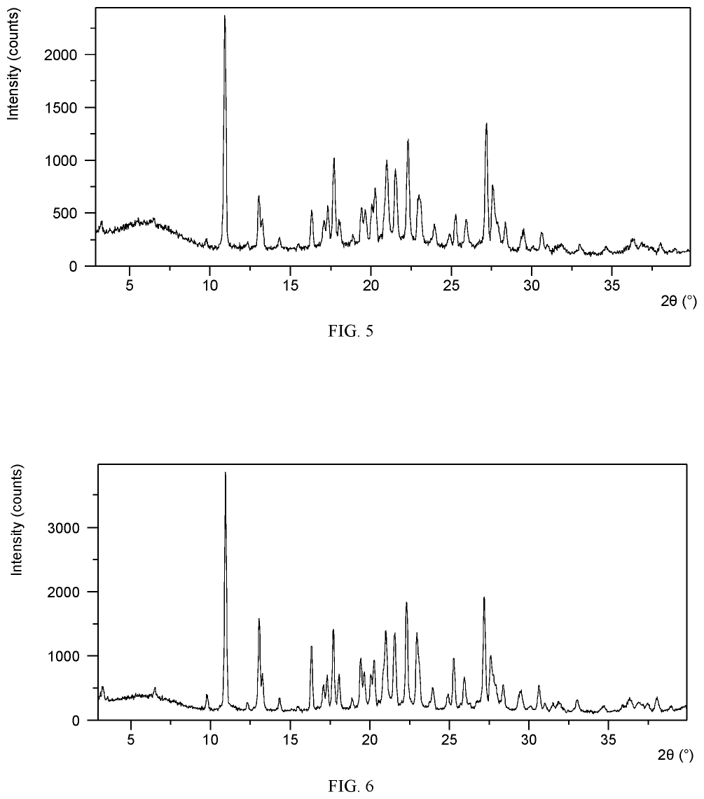

508.9 mg of upadacitinib freebase was weighed into a 20-mL glass vial, then 15 mL of n-hexane and 0.15 mL of acetic acid were added. The sample was stirred at room temperature for about 3 days. Then 0.1 mL of acetic acid was added with stirring at room temperature for about 3 days. The solid was obtained by isolation and washed twice with 3 mL of n-hexane. Then the solid was dried under forced air convection at 25° C. for 5 hours and confirmed to be Form CSI. The XRPD data are listed in Table 6, and the XRPD pattern is substantially as depicted in FIG. 5. There is 18.1 wt % of acetic acid in the obtained solid determined by HPLC.

TABLE 6

| 2θ | d spacing | Intensity % |
|---|---|---|
| 10.93 | 8.10 | 100.00 |
| 13.00 | 6.81 | 22.10 |
| 13.23 | 6.69 | 12.52 |
| 16.29 | 5.44 | 17.07 |
| 17.09 | 5.19 | 12.30 |
| 17.30 | 5.12 | 19.57 |
| 17.70 | 5.01 | 40.77 |
| 18.04 | 4.92 | 11.83 |
| 19.42 | 4.57 | 18.43 |
| 19.65 | 4.52 | 17.44 |
| 20.03 | 4.43 | 18.72 |
| 20.28 | 4.38 | 26.63 |
| 20.98 | 4.23 | 40.18 |
| 21.48 | 4.14 | 34.36 |
| 22.29 | 3.99 | 48.55 |
| 22.91 | 3.88 | 22.18 |
| 23.95 | 3.72 | 11.96 |
| 25.28 | 3.52 | 15.05 |
| 25.89 | 3.44 | 13.67 |
| 27.17 | 3.28 | 57.90 |
| 27.54 | 3.24 | 28.79 |
| 28.35 | 3.15 | 13.51 |

### Example 7 Preparation of Form CSI

1.5154 g of upadacitinib freebase was weighed into a 100-mL glass bottle, and then 50 mL of n-hexane and 1 mL of acetic acid were added. The system was stirred at room temperature for 1 day, and then the solid was isolated and dried under forced air convection at 25° C. for 18.5 hours. The obtained solid was confirmed to be Form CSI. The XRPD data are listed in Table 7, and the XRPD pattern is substantially as depicted in FIG. 6. There is 23.1 wt % of acetic acid in the obtained solid tested by HPLC.

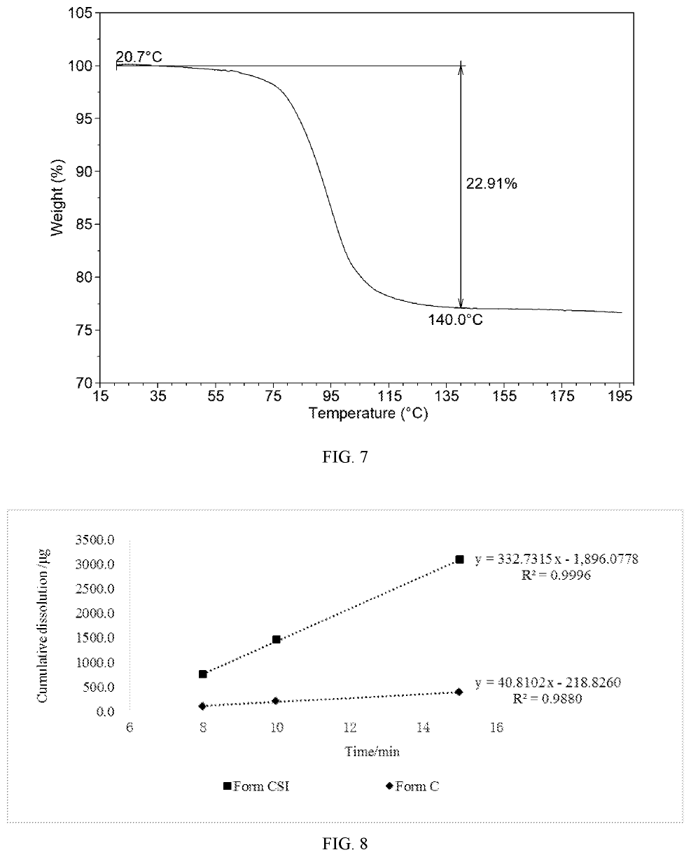

The TGA curve of Form CSI is as depicted in FIG. 7, which shows about 22.9% weight loss when heated to 140° C., corresponding to the loss of acetic acid during heating.

TABLE 7

| 2θ | d spacing | Intensity % |
|---|---|---|
| 10.93 | 8.10 | 100.00 |
| 13.04 | 6.79 | 38.64 |
| 13.25 | 6.68 | 14.97 |
| 16.31 | 5.43 | 27.30 |
| 17.07 | 5.19 | 10.50 |
| 17.31 | 5.12 | 14.99 |
| 17.69 | 5.01 | 33.97 |
| 18.06 | 4.91 | 15.47 |
| 19.41 | 4.57 | 21.81 |
| 19.66 | 4.52 | 15.94 |
| 20.04 | 4.43 | 14.91 |
| 20.26 | 4.38 | 21.21 |
| 21.02 | 4.23 | 31.50 |
| 21.54 | 4.13 | 31.78 |
| 22.32 | 3.98 | 44.97 |
| 22.91 | 3.88 | 28.32 |
| 23.09 | 3.85 | 19.71 |
| 25.25 | 3.53 | 22.00 |
| 25.93 | 3.44 | 14.28 |
| 27.18 | 3.28 | 48.20 |
| 27.56 | 3.24 | 22.85 |
| 28.36 | 3.15 | 11.21 |
| 30.60 | 2.92 | 10.86 |

### Example 8 Kinetic Solubility of Form CSI

The solubility of Form C was disclosed in WO2017066775A1. In order to have a comparison with Form C, solubility of Form CSI was measured. Saturated solutions of Form CSI of the present disclosure were prepared with pH7.4 PBS, pH6.5 FaSSIF and pH5.0 FeSSIF at 25° C. or 37° C. After equilibration for 24 h, 34 h and 48 h, the saturated solutions were obtained by filtration. The concentrations of compound I in the saturated solutions were measured by high performance liquid chromatography (HPLC) and the results are listed in Table 8.

TABLE 8

| | Solubility | | | |
|---|---|---|---|---|
| | Form CSI | | | |
| | 24 h | 34 h | 48 h | Form C |
| Media | (mg/mL) | (mg/mL) | (mg/mL) | (mg/mL) |
| pH 7.4, 25° C. | 0.79 | 0.71 | 0.66 | 0.19 |
| FaSSIF (pH 6.5, 37° C.) | 3.8 | 4.1 | 4.1 | 0.22 |
| FeSSIF (pH 5.0, 37° C.) | 3.1 | 3.0 | 3.1 | 0.47 |

The results show that the solubility of Form CSI is higher than that of Form C in pH7.4 PBS, pH6.5 FaSSIF and pH5.0 FeSSIF.

### Example 9 Intrinsic Dissolution Rate of Form CSI

100 mg of Form CSI and Form C in WO2017066775A1 were added into the cavity of the die, and then compressed at 1.5 KN and held for 0.5 minute. The whole pellet was transferred to a dissolution apparatus to test the intrinsic dissolution rate. Dissolution method is shown in Table 9, dissolution profile is presented in FIG. 8 and dissolution data are presented in Table 10. The slope (in μg/min) of the regression line was calculated according to the data within 8-15 minutes. Intrinsic dissolution rate (IDR) was further calculated according to the slope (in μg/min/cm²). IDR results are presented in Table 11.

TABLE 9

| | |
|---|---|
| Instrument | Agilent 708DS |
| Medium | pH 6.8 PBS |
| Volume | 900 mL |
| Speed | 100 rpm |
| Temperature | 37° C. |
| Sampling Time | 8, 10, 15 min |
| Supplement medium | No |

TABLE 10

Cumulative dissolution (μg) pH 6.8

| Time | | Form C in |
|---|---|---|
| (min) | Form CSI | WO2017066775A1 |
| 8 | 746.1 | 94.7 |
| 10 | 1458.8 | 207.4 |
| 15 | 3087.0 | 388.1 |

TABLE 11

| Solid Form | Slope (μg/min) | IDR (μg/min/cm²) |
|---|---|---|
| Form CSI | 332.7315 | 665.4630 |
| Form C in WO2017066775A1 | 40.8102 | 81.6204 |

The results show that IDR of Form CSI is over 8 times that of Form C in WO2017066775A1.

### Example 10 Stability of Form CSI

Solid samples of Form CSI in the present disclosure were sealed and stored under different conditions of 4° C., 25° C./60% RH, 40° C./75% RH, and 60° C./75% RH. Crystalline form was checked by XRPD before and after storage. The results are shown in Table 12.

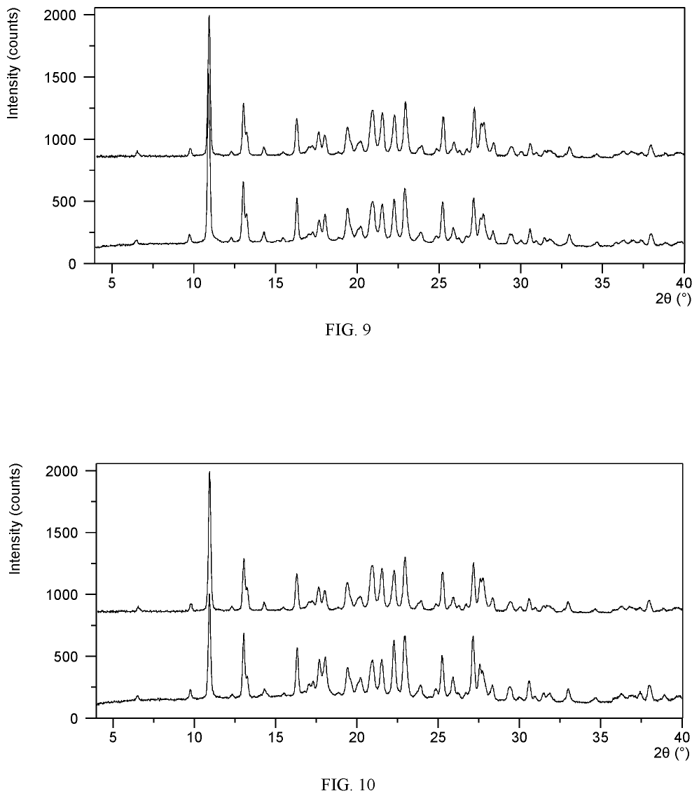

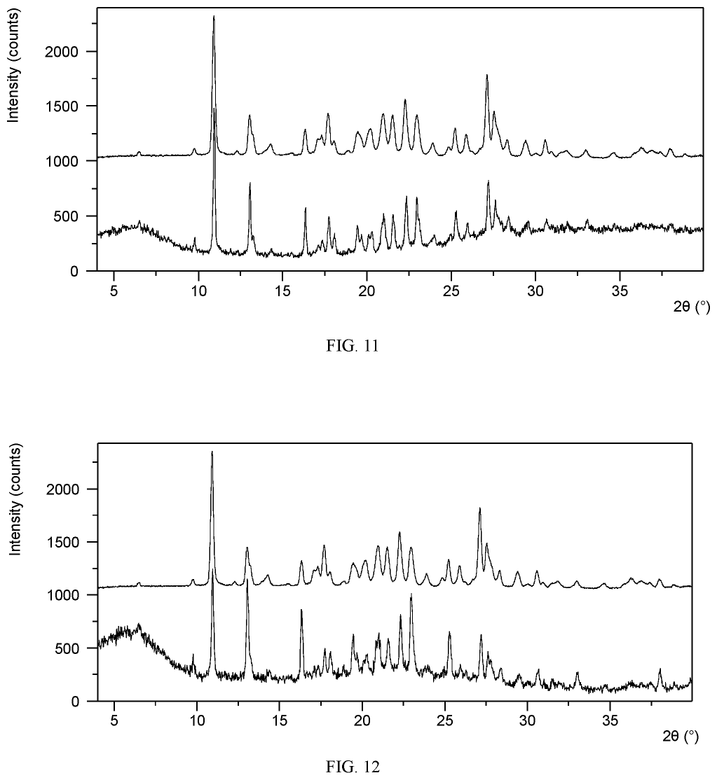

TABLE 12

| Condition | Time | | Solid Form | XRPD Overlay |
|---|---|---|---|---|
| 4° C. | 6 | months | Form CSI | FIG. 9 |
| 25° C./60% RH | 6 | months | Form CSI | FIG. 10 |
| 40° C./75% RH | 2 | weeks | Form CSI | FIG. 11 |
| 60° C./75% RH | 2 | weeks | Form CSI | FIG. 12 |

The results show that Form CSI kept stable for at least 6 months under the conditions of 4° C. and 25° C./60% RH. It shows that Form CSI has good stability under long-term stability condition. Form CSI kept stable for at least 2 weeks under the conditions of 40° C./75% RH and 60° C./75% RH. It shows that Form CSI also has good stability under more stress conditions.

### Example 11 Mechanical Stability of Form CSI

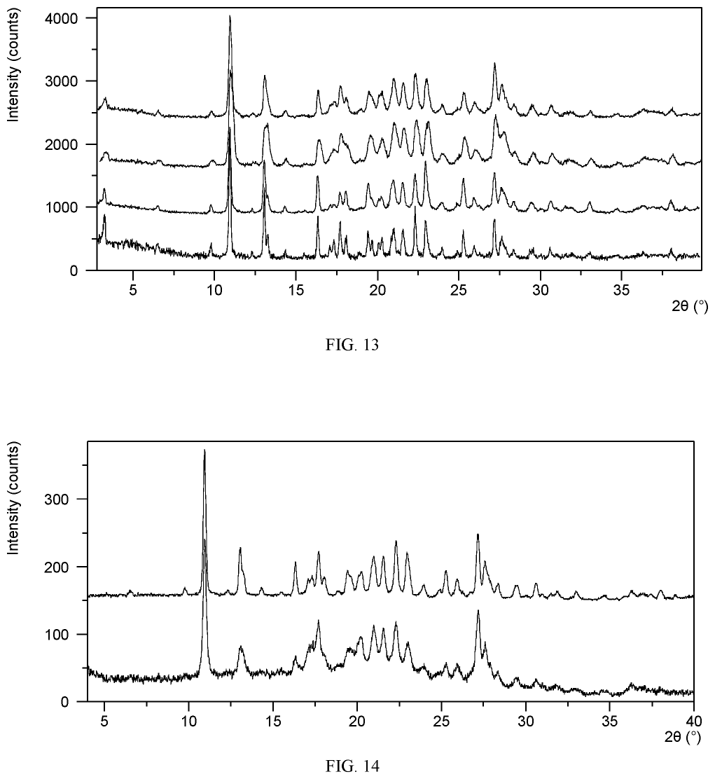

20 mg of Form CSI was weighed into the die of 16 mm round tooling (IDR punch), and tableted by ENERPAC manual tablet press with 3 kN, 7 kN, and 14 kN pressure. Crystalline forms before and after tableting were checked by XRPD and the results are presented in FIG. 13. The results show that the crystalline state of Form CSI in the present disclosure does not change and the crystallinity of Form CSI remains substantially unchanged after tableting.

A small amount of Form CSI was grounded manually for 5 minutes in a mortar. Crystalline forms before and after grinding were checked by XRPD and the results are presented in FIG. 14. The results show that the crystalline state of Form CSI in the present disclosure does not change and the crystallinity of Form CS I remains substantially unchanged after grinding.

### Example 12 Particle Size Distribution of Form CSI

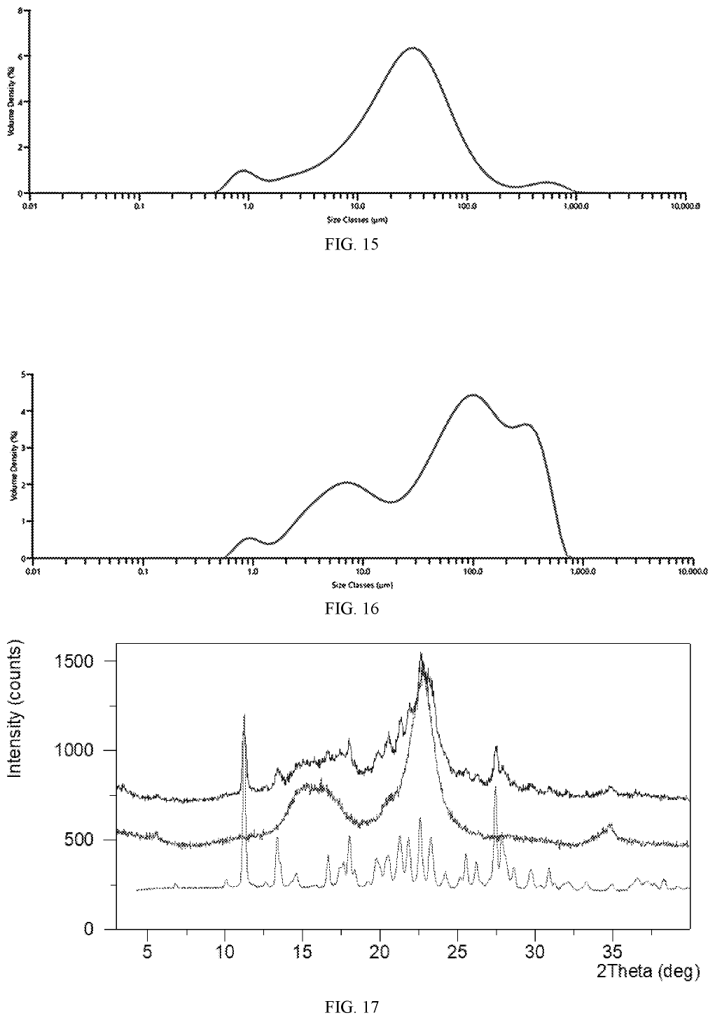

10-30 mg of Form CSI or Form C in WO2017066775A1 were mixed with about 5 mL of Isopar G (containing 0.2% lecithin). The mixture was mixed thoroughly and transferred into the Hydro MV dispersing device. The experiment started when the obscuration is in an appropriate range. The particle size distribution was tested after 30 seconds of ultrasound. The particle size distribution (PSD) patterns are substantially as depicted in FIG. 15 (Form CSI) and FIG. 16 (Form C). The results show that Form CSI has a more uniform particle size distribution when compared with Form C in WO2017066775A1.

### Example 13 Yield of Form CSI

Form C in WO2017066775A1: 1.5 g of upadacitinib freebase was dissolved into 47.5 mL of ethanol, and the solution was filtered into a 500-mL reactor. 150 mL of water was added into the reactor slowly with stirring at 6° C. After being stirred overnight, 1.13 g of solid was isolated, corresponding to a yield of 79.0% (in terms of upadacitinib freebase).

Form CSI: 1.5 g of upadacitinib freebase, 40 mL of n-hexane, and 0.4 mL of acetic acid were added into a 100-mL glass bottle. The sample was stirred at room temperature for about 5 days, then another 0.1 mL of acetic acid was added, and the system was stirred at room temperature for about another 2 days. The solid was isolated and dried under vacuum at 25° C. for about 1 h, and then 1.74 g of Form CSI was obtained, corresponding to a yield of 96.8% (in terms of upadacitinib freebase). The TGA curve of the obtained solid shows about 22.4% weight loss when heated to 150° C.

The results show that the yield of Form CSI is higher than that of Form C in WO2017066775A1.

### Example 14 Compressibility of Form CSI

An ENERPAC manual tablet press was used for compression. 80 mg of Form CSI of the present disclosure and prior art Form C were weighed and added into the dies of a round tooling (ensuring the isotropy of tables), compressed at 10 KN manually, and then stored at room temperature for 24 h until complete elastic recovery. Hardness (H) was tested with an intelligent tablet hardness tester. Diameter (D) and thickness (L) were tested with a caliper. Tensile strengths of the Form CSI of the present disclosure and Form C in WO2017066775A1 were calculated with the following formula: T=2H/πDL*9.8. Under a certain force, the greater the tensile strength, the better the compressibility. The results are presented in Table 13 and Table 14.

TABLE 13
Form CSI

| No. | 1 | 2 | 3 |
|---|---|---|---|
| Thickness (mm) | 2.33 | 2.22 | 2.25 |
| Diameter (mm) | 6.01 | 6.01 | 6.01 |
| Hardness (kgf) | 3.53 | 3.06 | 3.43 |
| Tensile strength (MPa) | 1.57 | 1.43 | 1.58 |

| | |
|---|---|
| Mean tensile strength (MPa) | 1.53 |

TABLE 14
Form C in WO2017066775A1

| No. | 1 | 2 | 3 |
|---|---|---|---|
| Thickness (mm) | 2.14 | 2.18 | 2.07 |
| Diameter (mm) | 6.00 | 6.01 | 6.00 |
| Hardness (kgf) | 0.84 | 1.70 | 1.70 |
| Tensile strength (MPa) | 0.41 | 0.81 | 0.85 |

| | |
|---|---|
| Mean tensile strength (MPa) | 0.69 |

The results indicate that Form CSI has better compressibility than Form C in WO2017066775A1.

### Example 15 Preparation of Form CSI Drug Product

Form CSI of the present disclosure was made into tablet according to the formulation in Table 15 and process in Table 16. Crystalline forms before and after formulation were checked by XRPD, and the XRPD pattern overlay is substantially as depicted in FIG. 17. The results show that Form CSI has good physical stability before and after formulation process.

TABLE 15

| | | | | |
|---|---|---|---|---|
| No. | Component | mg/unit | % (w/w) | Function |

Intragranular material

| | | | | |
|---|---|---|---|---|
| 1 | Upadacitinib freebase | 30.0 | 30.0 | API |
| 2 | Microcrystalline Cellulose | 60.0 | 60.0 | Filler |
| | (PH 101) | | | |
| 3 | Hypromellose (E5) | 3.0 | 3.0 | Binder |
| 4 | Crospovidone XL | 6.0 | 6.0 | Disintegration |
| 5 | Magnesium stearate (5712) | 0.5 | 0.5 | Lubricant |

Extragranular material

| | | | | |
|---|---|---|---|---|
| 6 | Magnesium stearate (5712) | 0.5 | 0.5 | Lubricant |

| | | | |
|---|---|---|---|
| Total | 100.00 | 100.00 | NA |

TABLE 16

| Stage | Procedure |
|---|---|
| Pre-blending | According to the formulation, No. 1-5 materials were |
| | weighted into a PE bag and blended manually for 2 min. |
| Sifting | The pre-blend powder was sieved through a 35 mesh sieve |
| | into a PE bag and blended for 1 min. |
| Simulated | The tablets were prepared by compressing with a manual |
| dry | single punch tablet press. (Model: ENERPAC; Die: φ20 |
| granulation | mm round; Weight: 500 mg ± 10.0 mg; Pressure: 5 kN ± |
| | 1 kN) |
| Pulverize | The tablets were pulverized and sieved through 20 mesh |
| | sieves. |
| Final | The extra-granular material and pulverized particles were |
| blending | blended manually for 2 minutes in a PE bag. |
| Tableting | The tablets were prepared by compressing with a single |
| | punch manual tablet press. (Model: ENERPAC; Die: φ7 |
| | mm round; Weight: 100.0 mg ± 2.0 mg; Force: 5 kN ± |
| | 1 kN) |
| Package | The tablets were packed in 35 cc HDPE bottles, one tablet |
| | per bottle with 1 g of desiccant. |

### Example 16 Preparation of Form CSI Drug Product

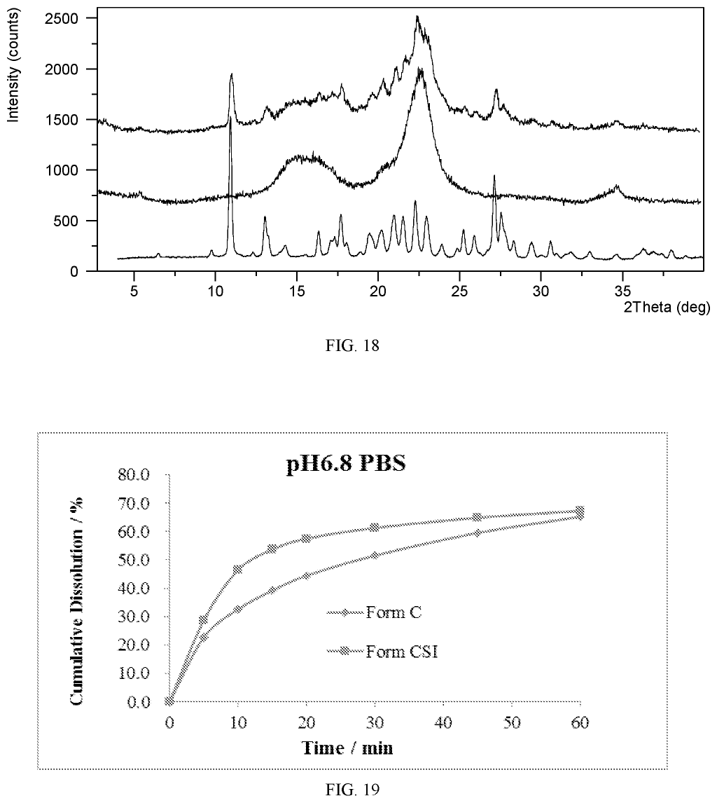

Form CSI of the present disclosure and Form C in WO2017066775A1 were made into tablet according to the formulation in Table 17 and process in Table 18. Crystalline forms before and after formulation were checked by XRPD, and the comparison of XRPD patterns are substantially as depicted in FIG. 18. The results show that Form CSI remains unchanged after formulation process.

TABLE 17

| | | | | |
|---|---|---|---|---|
| No. | Component | mg/unit | % (w/w) | Function |

Intragranular material

| | | | | |
|---|---|---|---|---|
| 1 | Upadacitinib freebase | 30.0 | 30.0 | API |
| 2 | Microcrystalline Cellulose | 60.0 | 60.0 | Filler |
| | (PH 101) | | | |
| 3 | Crospovidone XL | 9.0 | 9.0 | Disintegration |
| 4 | Magnesium stearate (5712) | 0.5 | 0.5 | Lubricant |

Extragranular material

| | | | | |
|---|---|---|---|---|
| 5 | Magnesium stearate (5712) | 0.5 | 0.5 | Lubricant |

| | | | |
|---|---|---|---|
| [symbol] | 100.00 | 100.00 | NA |

Note:
* The formulation is the same for both Form CSI or Form C in WO2017066775A1.

TABLE 18

| Stage | Procedure |
|---|---|
| Pre-blending | According to the formulation, No. 1-4 materials were |
| | weighted into a PE bag and blended manually for 2 min. |
| Sifting | The pre-blend powder was passed through 40 mesh sieves |
| | into a PE bag and blended for 1 min. |
| Simulated | The tablets were prepared by compressing with a manual |
| dry | single punch tablet press. (Model: ENERPAC; Die: φ20 |
| granulation | mm round; Weight: 500 mg ± 10.0 mg; Force: 5 kN ± |
| | 1 kN) |
| Mill | The tablets were crushed into the granules, then passed |
| | through 20 mesh sieves. |
| Final | The extra-granular material and sieved granules were |
| blending | blended manually for 2 minutes in a PE bag. |
| Compression | The tablets were prepared by compressing with a single |
| | punch manual tablet press. (Model: ENERPAC; Die: φ7 |
| | mm round; Weight: 100.0 mg ± 2.0 mg; Force: 5 kN ± |
| | 1 kN) |
| Package | The tablets were packed in 35 cc HDPE bottle, one tablet |
| | per bottle with 1 g desiccant. |

### Example 17 Dissolution Profile of Form CSI Drug Product

In vitro dissolution test was performed on drug products of Form CSI and Form C in WO2017066775A1 obtained from example 16. Dissolution method according to Chinese Pharmacopoeia 2015<0931> was used, and the conditions are listed in Table 19. Dissolution results of both drug products are presented in Table 20 and their dissolution profiles are shown in FIG. 19.

TABLE 19

| | |
|---|---|
| Instrument | SOTAX 708DS |
| Method | Paddle |
| Dosage | 30 mg |
| Medium | pH 6.8 PBS |
| Volume | 900 mL |
| Speed | 75 rpm |
| Temperature | 37° C. |
| Sampling Time | 5, 10, 15, 20, 30, 45, 60 minutes |
| Supplement of | No |
| medium | |

TABLE 20

Cumulative dissolution (%)

| Time | Form C in | |
|---|---|---|
| (min) | WO2017066775A1 | Form CSI |
| 0 | 0.0 | 0.0 |
| 5 | 22.7 | 28.7 |
| 10 | 32.6 | 46.4 |
| 15 | 39.2 | 53.6 |
| 20 | 44.4 | 57.5 |
| 30 | 51.5 | 61.3 |
| 45 | 59.5 | 64.9 |
| 60 | 65.2 | 67.3 |

The results show that the dissolution rate of Form CSI is obviously better than that of Form C in WO2017066775A1, which indicates that Form CSI has better bioavailability than Form C in WO2017066775A1.

The examples described above are only for illustrating the technical concepts and features of the present disclosure and intended to make those skilled in the art being able to understand the present disclosure and thereby implement it, and should not be concluded to limit the protective scope of the present disclosure. Any equivalent variations or modifications according to the spirit of the present disclosure should be covered by the protective scope of the present disclosure.

1. A crystalline Form CSI of upadacitinib exhibiting an X-ray powder diffraction pattern comprising characteristic peaks at 2theta values of 10.9°±0.2°, 13.0°±0.2° and 22.9°±0.2° using CuKα radiation.

2. The crystalline Form CSI according to claim 1, wherein the X-ray powder diffraction pattern shows one or two or three characteristic peaks at 2theta values of 27.2°±0.2°, 22.3°±0.2° and 16.3°±0.2° using CuKα radiation.

3. The crystalline Form CSI according to claim 1, wherein the X-ray powder diffraction pattern shows one or two or three characteristic peaks at 2theta values of 21.0°±0.2°, 21.5°±0.2° and 25.3°±0.2° using CuKα radiation.

4. A process for preparing crystalline Form CSI of upadacitinib, wherein the process comprises:
mixing upadacitinib freebase, acetic acid and organic solvents, crystallizing by stirring to obtain a solid.

5. The process according to claim 4, wherein said organic solvents are ethers and alkanes.

6. The process according to claim 5, wherein said ether is methyl tert-butyl ether, said alkane is n-hexane, n-heptane and a mixture thereof.

7. A pharmaceutical composition, wherein the pharmaceutical composition comprises a therapeutically effective amount of crystalline Form CSI according to claim 1 and a pharmaceutically acceptable carrier, diluent, or excipient.

8. (canceled)

9. A method of treating rheumatoid arthritis comprising administering to a subject in need thereof a therapeutically effective amount of crystalline Form CSI of upadacitinib according to claim 1.
